(19) United States
(12) Patent Application Publication
(10) Pub. No.: US 2025/0346621 A1
(43) Pub. Date: Nov. 13, 2025

(54) ASCAROSIDE SALTS

(71) Applicant: Ascribe Bioscience Inc., Ithaca, NY (US)

(72) Inventors: Jay Farmer, Ithaca, NY (US); Gabriel Wilmoth, Capel Hill, NC (US)

(21) Appl. No.: 18/864,686

(22) PCT Filed: May 10, 2023
(86) PCT No.: PCT/US2023/021731

§ 371 Date: Nov. 11, 2024

Related U.S. Application Data
(60) Provisional application No. 63/341,332, filed on May 12, 2022.

(51) Int. Cl. C07H 15/10; A01N 43/16; A01P 21/00

(52) U.S. Cl. CPC C07H 15/10; A01N 43/16; A01P 21/00

(57) ABSTRACT

This application relates to salts of ascarosides, which can provide various benefits over corresponding ascarosides. For example, certain ascaroside salts can provide beneficial nutrients to a plant to which or near which the salt is applied. Compositions and methods involving such ascaroside salts are also provided herein.

### CROSS-REFERENCE TO RELATED APPLICATIONS

This application claims priority to U.S. Provisional Patent Application Ser. No. 63/341,332entitled “Ascaroside Salts” filed on May 12, 2022, which is incorporated herein by reference.

### FIELD OF THE INVENTION

This application generally relates to agrichemical compounds, compositions and methods of treating plants to promote resistance to pathogens.

### BACKGROUND OF THE INVENTION

Ascaroside natural products are secondary metabolites produced by nematodes. A large number of structurally diverse ascarosides have been identified in nature and the molecules are believed to function as an evolutionarily conserved chemical language used by nematodes to control many aspects of their development. Ascarosides are also perceived by other organisms and have been demonstrated to have a range of effects on numerous organisms including bacteria, fungi, plants, and mammals including humans. Ascarosides hold potential as human medicines, agrichemicals and products for other diverse and valuable applications.

Ascaroside treatments have been demonstrated to show efficacy in increasing plant resistance to certain pathogens and/or in inducing and priming plant defense responses (which can inhibit pathogen growth and/or infestation) when applied to the plant. By activating and/or priming plants' innate defenses, ascarosides can thereby prevent proliferation of pathogens and/or protect crops from the damaging effects caused by diverse pathogens.

It would be useful to provide ascarosides in other forms to modify the physical properties of the compounds and, further, to provide additional benefits to plants or other organisms to which ascarosides are applied or administered.

### SUMMARY OF THE INVENTION

The disclosure provides compositions and methods for ascaroside delivery. In certain embodiments, such compositions and methods are useful in an agrichemical context. Such compositions and methods relate generally to salts of ascarosides which, in some embodiments, can provide various benefits to an organism (e.g. a plant, animal or microbe) to which such a salt is applied or administered.

The present disclosure includes, without limitation, the following embodiments.

Embodiment 1: A salt of the formula [A-(C(O)O)⁻]ₚM^{p+}, wherein: A is an ascaroside moiety; M is a metal or ammonium cation; and p is an integer from 1 to 3, and wherein, when p is 2 or 3, each A can be the same or different.

Embodiment 2: The salt of Embodiment 1, wherein p is 1.

Embodiment 3: The salt of Embodiment 1, wherein p is 2.

Embodiment 4: The salt of Embodiment 1, wherein p is 3.

Embodiment 5: The salt of any one of Embodiments 1-4, wherein M is a metal.

Embodiment 6: The salt of Embodiment 5, wherein the metal is selected from the group consisting of potassium (K), calcium (Ca), magnesium (Mg), zinc (Zn), manganese (Mn), iron (Fe), copper (Cu), molybdenum (Mo), and nickel (Ni).

Embodiment 7: The salt of Embodiment 6, wherein the metal is selected from the group consisting of copper (Cu), zinc (Zn), manganese (Mn), and iron (Fe).

Embodiment 8: The salt of any one of Embodiments 1-4, wherein M is an ammonium cation or an analogue thereof.

Embodiment 9: A salt comprising at least one boron atom coordinated to an ascaroside (A).

Embodiment 10: The salt of any of Embodiments 1-9, wherein A has the structure (I)

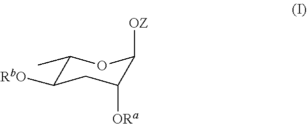

where: Z is an optionally substituted C₂₋₄₀ aliphatic group, and each of R^{a} and R^{b} is independently —H, or an optionally substituted moiety selected from the group consisting of: C₁₋₂₀ aliphatic, C₁₋₂₀ acyl, C₁₋₂₀ heteroaliphatic, aryl, heteroaryl, a hydroxyl protecting group, a phosphorous-linked functional group, a sulfur-linked functional group, a silicon-linked functional group, a C₂₋₂₀ carbonate (e.g. -a moiety —C(O)OR^{c}), a C₂₋₂₀ carbamate (e.g. -a moiety —C(O)N(R^{c})₂), a C₂₋₂₀ thioester (e.g. a moiety) —C(S)R^{c}), a C₂₋₂₀ thiocarbonate (e.g. a moiety —C(S)OR^{c}), a C₂₋₂₀ dithiocarbonate (e.g. a moiety —C(S)SR^{c}), a C₁₋₂₀ thiocarbamate (e.g. a moiety —C(S)N(R^{c})₂), a sugar moiety, a peptide, a polymer chain, or a linkage via a bond or a carbon-containing linker moiety to another ascaroside molecule. Where R^{c} is independently at each occurrence selected from —H, optionally substituted C₁₋₁₂ aliphatic, optionally substituted C₁₋₁₂ heteroaliphatic, optionally substituted aryl, optionally substituted heteroaryl, a polymer chain, or a linkage via a bond or a carbon-containing linker moiety to another ascaroside molecule, and where R^{a} and R^{b} may be taken together to form an optionally substituted ring, optionally containing one or more heteroatoms, and optionally containing one or more sites of unsaturation.

Embodiment 11: The salt of Embodiment 10, wherein Z is selected from the group consisting of: i. —CH(CH₃)—R¹, where R¹ is an optionally substituted C₁₋₄₀ aliphatic group; ii. —CH(CH₃)—(CH₂)ₙ—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule; iii. —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule; iv. —CH(CH₃)—(CH₂)ₙ—CH(OH)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule; v. —CH(CH₃)—(CH₂)ₙ—C(O)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule; vi. —(CH₂)ₙ—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule; vii. —(CH₂)ₙ—CH═CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule; viii. —(CH₂)ₙ—CH(OH)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule; and ix. —(CH₂)ₙ—C(O)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule.

Embodiment 12: The salt of Embodiment 10 or 11, wherein R^{a} and R^{b} are each —H.

Embodiment 13: The salt of any one of Embodiments 10-12, wherein Z is —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

Embodiment 14: The salt of any one of Embodiment 1-13, wherein A is ascr #18.

Embodiment 15: A salt of the formula:

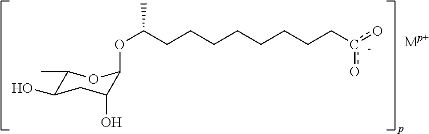

wherein M is a metal or ammonium cation; and p is an integer from 1 to 3.

Embodiment 16: A composition comprising the salt of any of Embodiments 1-14.

Embodiment 17: The composition of Embodiment 16, in solid form.

Embodiment 18: The composition of Embodiment 17, wherein the solid form comprises powder or granules.

Embodiment 19: The composition of Embodiment 16, in liquid form.

Embodiment 20: The composition of Embodiment 19, wherein the liquid form is a sprayable formulation.

Embodiment 21: The composition of any of Embodiments 16-20, further comprising one or more additional components selected from the group consisting of surfactants, including emulsifiers, dispersants, foam-formers, colorants, processing aids, lubricants, fillers, reinforcements, flame retardants, light stabilizers, ultraviolet radiation absorbers, weather stabilizers, plasticizers, release agents, perfumes, heat-retaining additives (e.g., silica), cross-linking agents, antioxidants, anti-foaming agents, buffers, pH modifiers, compatibility agents, drift control additives, extenders/stickers, tackifiers, plant penetrants, safeners, spreaders, and wetting agents.

Embodiment 22: A method of enhancing the physical properties of an ascaroside and providing one or more nutrients to a plant, comprising: contacting a plant or portion thereof or soil surrounding the plant or portion thereof with the salt of any of Embodiments 1-15 or the composition of any of Embodiments 16-21.

Embodiment 23: The method of Embodiment 22, wherein the portion thereof is selected from the group consisting of root, stem, leaf, seed, and flower.

Embodiment 24: The method of Embodiment 22 or 23, wherein the plant is a vegetable or fruit plant.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise. Other aspects and advantages of the present disclosure will become apparent from the following.

### Definitions

In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. As used in this application, the term “comprise” and variations of the term, such as “comprising” and “comprises,” are not intended to exclude other additives, components, integers or steps.

As used herein, the terms “about” and “approximately” are used as equivalents. Unless otherwise stated, the terms “about” and “approximately” may be understood to permit standard variation as would be understood by those of ordinary skill in the art. Where ranges are provided herein, the endpoints are included. Any numerals used in this application with or without about/approximately are meant to cover any normal fluctuations appreciated by one of ordinary skill in the relevant art. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

Certain salts provided herein can comprise one or more asymmetric centers, and thus can exist in various stereoisomeric forms, e.g., enantiomers and/or diastereomers. Thus, inventive salts and compositions thereof may be in the form of an individual enantiomer, diastereomer or geometric isomer, or may be in the form of a mixture of stereoisomers. In certain embodiments, inventive salts and compositions described herein can comprise enantiopure salts. In certain other embodiments, mixtures of enantiomers or diastereomers are provided.

Furthermore, certain salts as described herein may have one or more double bonds that can exist as either a Z or E isomer, unless otherwise indicated. The salts can be provided as individual isomers substantially free of other isomers and alternatively, as mixtures of various isomers, e.g., racemic mixtures of enantiomers.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the disclosure. For instance, a salt may, in some embodiments, be provided substantially free of one or more corresponding stereoisomers, and may also be referred to as “stereochemically enriched.”

Where a particular enantiomer is preferred, it may, in some embodiments be provided substantially free of the opposite enantiomer, and may also be referred to as “optically enriched.” “Optically enriched,” as used herein, means that the salt is made up of a significantly greater proportion of one enantiomer. In certain embodiments the salt is made up of at least about 90% by weight of an enantiomer. In some embodiments the salt is made up of at least about 95%, 97%, 98%, 99%, 99.5%, 99.7%, 99.8%, or 99.9% by weight of an enantiomer. In some embodiments the enantiomeric excess of provided salts is at least about 90%, 95%, 97%, 98%, 99%, 99.5%, 99.7%, 99.8%, or 99.9%. In some embodiments, enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972), which are incorporated herein by reference.

The terms “halo” and “halogen” as used herein refer to an atom selected from fluorine (fluoro, —F), chlorine (chloro, —Cl), bromine (bromo, —Br), and iodine (iodo, —I).

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-30 carbon atoms. In certain embodiments, aliphatic groups contain 1-12 carbon atoms. In certain embodiments, aliphatic groups contain 1-8 carbon atoms. In certain embodiments, aliphatic groups contain 1-6 carbon atoms. In some embodiments, aliphatic groups contain 1-5 carbon atoms, in some embodiments, aliphatic groups contain 1-4 carbon atoms, in yet other embodiments aliphatic groups contain 1-3 carbon atoms, and in yet other embodiments aliphatic groups contain 1-2 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “heteroaliphatic” or “heteroaliphatic group,” as used herein, denotes an aliphatic group where one or more carbon or hydrogen atoms are replaced by a heteroatom (e.g. oxygen, nitrogen, sulfur, phosphorous, boron, etc.).

The term “unsaturated,” as used herein, means that a moiety has one or more double or triple bonds.

The term “alkyl,” as used herein, refers to saturated, straight- or branched-chain hydrocarbon radicals derived from an aliphatic moiety containing between one and six carbon atoms by removal of a single hydrogen atom. Unless otherwise specified, alkyl groups contain 1-12 carbon atoms. In certain embodiments, alkyl groups contain 1-8 carbon atoms. In certain embodiments, alkyl groups contain 1-6 carbon atoms. In some embodiments, alkyl groups contain 1-5 carbon atoms, in some embodiments, alkyl groups contain 1-4 carbon atoms, in yet other embodiments alkyl groups contain 1-3 carbon atoms, and in yet other embodiments alkyl groups contain 1-2 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

The term “alkenyl,” as used herein, denotes a monovalent group derived from a straight-or branched-chain aliphatic moiety having at least one carbon-carbon double bond by the removal of a single hydrogen atom. Unless otherwise specified, alkenyl groups contain 2-12 carbon atoms. In certain embodiments, alkenyl groups contain 2-8 carbon atoms. In certain embodiments, alkenyl groups contain 2-6 carbon atoms. In some embodiments, alkenyl groups contain 2-5 carbon atoms, in some embodiments, alkenyl groups contain 2-4 carbon atoms, in yet other embodiments alkenyl groups contain 2-3 carbon atoms, and in yet other embodiments alkenyl groups contain 2 carbon atoms. Alkenyl groups include, for example, ethenyl, propenyl, butenyl, 1-methyl-2-buten-1-yl, and the like.

The term “aryl” used alone or as part of a larger moiety as in “aralkyl”, “aralkoxy”, or “aryloxyalkyl”, refers to monocyclic and polycyclic ring systems having a total of five to 20 ring members, wherein at least one ring in the system is aromatic and wherein each ring in the system contains three to twelve ring members. The term “aryl” may be used interchangeably with the term “aryl ring”. In certain embodiments, “aryl” refers to an aromatic ring system which includes, but is not limited to, phenyl, biphenyl, naphthyl, anthracyl and the like, which may bear one or more substituents. Also included within the scope of the term “aryl”, as it is used herein, is a group in which an aromatic ring is fused to one or more additional rings, such as benzofuranyl, indanyl, phthalimidyl, naphthimidyl, phenantriidinyl, or tetrahydronaphthyl, and the like.

As described herein, salts as provided herein may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned are preferably those that result in the formation of stable or chemically feasible compounds/salts. The term “stable,” as used herein, refers to compounds or salts that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{○}; —(CH₂)₀₋₄OR^{○}; —O—(CH₂)₀₋₄C(O)OR^{○}; —(CH₂)₀₋₄CH(OR^{○})₂; —(CH₂)₀₋₄SR^{○}; —(CH₂)₀₋₄Ph, which may be substituted with R^{○}; —(CH₂)₀₋₄₀O(CH₂)₀₋₁Ph which may be substituted with R^{○}; —CH═CHPh, which may be substituted with R^{○}; —NO₂; —CN; —N₃; —(CH₂)₀₋₄N(R^{○})₂; —(CH₂)₀₋₄N(R^{○})C(O)R^{○}; —N(R⁵⁰¹ )C(S)R^{○}; —(CH₂)₀₋₄N(R^{○})C(O)NR^{○}₂; —N(R^{○})C(S)NR^{○}₂; —(CH₂)_{0- 4}N(R^{○})C(O)OR^{○}; —N(R^{○})N(R^{○})C(O)R^{○}; —N(R^{○})N(R^{○})C(O)NR^{○}₂; —N(R^{○})N(R^{○})C(O)OR^{○}; —(CH₂)₀₋₄C(O)R^{○}; —C(S)R^{○}; —(CH₂)₀₋₄C(O)OR^{○}; —(CH₂)₀₋₄C(O)N(R^{○})₂; —(CH₂)₀₋₄C(O)SR^{○}; —(CH₂)₀₋₄C(O)OSiR^{○}₃; —(CH₂)₀₋₄OC(O)R^{○}; —OC(O)(CH₂)₀₋₄SR—, SC(S)SR^{○}; —(CH₂)₀₋₄SC(O)R^{○}; —(CH₂)₀₋₄C(O)NR^{○}₂; —C(S)NR^{○}₂; —C(S)SR^{○}; —SC(S)SR⁵⁰¹ , —(CH₂)₀₋₄OC(O)NR^{○}₂; —C(O)N(OR^{○})R^{○}; —C(O)C(O)R^{○}; —C(O)CH₂C(O)R^{○}; —C(NOR^{○})R^{○}; —(CH₂)₀₋₄SSR^{○}; —(CH₂)₀₋₄S(O)₂R^{○}; —(CH₂)₀₋₄S(O)₂OR^{○}; —(CH₂)₀₋₄OS(O)₂R^{○}; —S(O)₂NR^{○}₂; —(CH₂)₀₋₄S(O)R^{○}; —N(R^{○})S(O)₂NR^{○}₂; —N(R^{○})S(O)₂R^{○}; —N(OR^{○})R^{○}; —C(NH)NR^{○}₂; —P(O)₂R^{○}; —P(O)R^{○}₂; —OP(O)R^{○}₂; —OP(O)(OR^{○})₂; SiR^{○}₃; —(C₁₋₄ straight or branched alkylene)O—N(R^{○})₂; or —(C₁₋₄ straight or branched alkylene)C(O)O—N(R^{○})₂, wherein each R^{○} may be substituted as defined below and is independently hydrogen, C₁₋₈ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{○}, taken together with their intervening atom(s), form a 3-12-membered saturated, partially unsaturated, or aryl mono- or polycyclic ring having 0-4 heteroatoms independently selected from nitrogen, oxygen, or sulfur, which may be substituted as defined below.

Suitable monovalent substituents on R^{○} (or the ring formed by taking two independent occurrences of R^{○} together with their intervening atoms), are independently halogen, —(CH₂)₀₋₂R^{●}, —(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₄C(O)N(R^{○})₂; —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR⁵⁷⁴ wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{○} include ═O and ═S.

Suitable divalent substituents on a saturated carbon atom of an “optionally substituted” group include the following: ═O, ═S, ═NNR^{*}₂, ═NNHC(O)R^{*}, ═NNHC(O)OR^{*}, ═NNHS(O)₂R^{*}, ═NR^{*}, ═NOR^{*}, —O(C(R^{*}₂))₂₋₃O—, or —S(C(R^{*}₂))₂₋₃S—, wherein each independent occurrence of R^{*} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{*}₂)₂₋₃O—, wherein each independent occurrence of R^{*} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{*} include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, C(O)C(O)R^{†}, C(O)CH₂C(O)R^{†}, —S(O)₂R⁺, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “substantially” refers to the qualitative condition of exhibiting total or near-total extent or degree of a characteristic or property of interest.

The convention of naming ascarosides by a several-letter prefix followed by a pound sign (#) and a number is sometimes used (for example ascr #18). This convention is used in the scientific literature and the skilled artisan will understand that each such name is associated with a specific chemical structure of known composition and will readily apprehend the structure of the molecule referred to using this naming convention. Unless otherwise indicated, all compound identifiers of this format used herein conform to the definitions described in the C. elegans Small Molecule Identifier Database (SMID-DB) maintained at http://www.smid-db.org.

The term “pathogen” refers to any bacterium, fungus, oomecyte, virus, nematode (e.g., cyst or root knot nematode) or insect with pathogenic effects on a plant.

### BRIEF DESCRIPTION OF THE DRAWINGS

The present teachings described herein will be more fully understood from the following description of various illustrative embodiments, when read together with the accompanying drawing. It should be understood that the drawing described below is for illustration purposes only and is not intended to limit the scope of the present teachings in any way. The foregoing and other objects, aspects, features, and advantages of the disclosure will become more apparent and may be better understood by referring to the following description taken in conjunction with the accompanying drawing, in which:

FIG. 1 shows a chart of the relative activity of ascr #18 vs. its zinc salt in an assay that measures protection of wheat plants from damage by the fungal pathogen Bipolaris sorokiniana.

### DETAILED DESCRIPTION OF THE INVENTION

This disclosure is directed to ascarosides in salt form (also referred to herein as “ascaroside salts”), as well as to compositions and methods involving such ascaroside salts. In some embodiments, use of an ascaroside in salt form can provide various formulation benefits as compared with the corresponding ascaroside (e.g., improved handling characteristics and/or simplified or improved incorporation within agrichemical or pharmaceutical compositions). In some embodiments, use of an ascaroside in certain salt forms can also provide benefits to plants to which the ascaroside salt is applied, beyond the benefits generally afforded by application of an ascaroside, e.g., providing micronutrients, macronutrients and/or other beneficial molecules.

Ascarosides are derivatives of the sugar ascarylose—a di-deoxy sugar lacking hydroxyl groups at its 3- and 6-positions. Ascarosides have the general structure shown in Formula I:

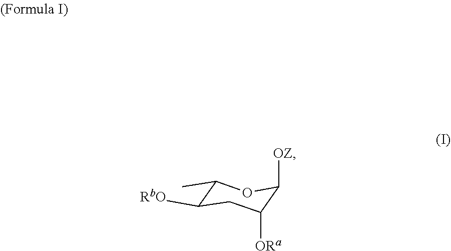

wherein:
Z is an optionally substituted C₂₋₄₀ aliphatic group, and
each of R^{a} and R^{b} is independently —H, or an optionally substituted moiety selected from the group consisting of: C₁₋₂₀ aliphatic, C₁₋₂₀ acyl, C₁₋₂₀ heteroaliphatic, aryl, heteroaryl, a hydroxyl protecting group, a phosphorous-linked functional group, a sulfur-linked functional group, a silicon-linked functional group, a C₂₋₂₀ carbonate (e.g., -a moiety —C(O)OR^{c}), a C₂₋₂₀ carbamate (e.g., -a moiety —C(O)N(R^{c})₂), a C₂₋₂₀ thioester (e.g., a moiety —C(S)R^{c}), a C₂₋₂₀ thiocarbonate (e.g., a moiety —C(S)OR^{c}), a C₂₋₂₀ dithiocarbonate (e.g., a moiety —C(S)SR^{c}), a C₁₋₂₀ thiocarbamate (e.g., a moiety —C(S)N(R^{c})₂), a sugar moiety, a peptide, a polymer chain, or a linkage via a bond or a carbon-containing linker moiety to another ascaroside molecule, where R^{c} is independently at each occurrence selected from —H, optionally substituted C₁₋₁₂ aliphatic, optionally substituted C₁₋₁₂ heteroaliphatic, optionally substituted aryl, optionally substituted heteroaryl, a polymer chain, or a linkage via a bond or a carbon-containing linker moiety to another ascaroside molecule, and where R^{a} and R^{b} may be taken together to form an optionally substituted ring, optionally containing one or more heteroatoms, and optionally containing one or more sites of unsaturation.

In certain embodiments, Z is:
(i) —CH(CH₃)—R¹, where R¹ is an optionally substituted C₁₋₄₀ aliphatic group;
(ii) —CH(CH₃)—(CH₂)ₙ—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(iii) —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(iv) —CH(CH₃)—(CH₂)ₙ—CH(OH)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(v) —CH(CH₃)—(CH₂)ₙ—C(O)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(vi) —(CH₂)ₙ—CO₂R², where n is an integer from 1 to 40, and R² is is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(vii) —(CH₂)ₙ—CH═CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(viii) —(CH₂)ₙ—CH(OH)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule; or
(ix) —(CH₂)ₙ—C(O)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule.

In certain embodiments, Z is:
(x) —CH(CH₃)—(CH₂)ₙ—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xi) —CH(CH₃)—(CH₂)ₙ—CH═CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xii) —CH(CH₃)—(CH₂)ₙ—CH(OH)—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xiii) —CH(CH₃)—(CH₂)ₙ—C(O)—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xiv) —(CH₂)ₙ—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xv) —(CH₂)ₙ—CH═CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xvi) —(CH₂)ₙ—CH(OH)—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule; or
(x) —(CH₂)ₙ—C(O)—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule.

In certain embodiments, R^{a} is —H.

In certain embodiments R^{b} is —H.

In certain embodiments, R^{a} and R^{b} are the same. In certain embodiments, R^{a} and R^{b} are both —H.

In certain embodiments, R^{a} and R^{b} are different. In certain embodiments, R^{a} is-H, and R^{b} is other than —H. In certain embodiments, R^{a} is other than —H and R^{b} is —H. In certain embodiments, R^{a} is —H and R^{b} is p-hydroxybenzoate. In certain embodiments, R^{a} is —H and R^{b} is indole-3-carboxylate. In certain embodiments, R^{a} is —H and R^{b} is (E)-2-methyl-2-butenoate. In certain embodiments, R^{a} is —H and R^{b} is picolinate. In certain embodiments, R^{a} is —H and R^{b} is nicotinate. In certain embodiments, R^{a} is —H and R^{b} is (R)-2-hydroxy-2-(4-hydroxyphenyl)ethyl)amino)-4-oxobutanoate. In certain embodiments, R^{a} is —H and R^{b} is 4-((4-hydroxyphenethyl)amino)-4-oxobutanoate.

In certain embodiments R^{a} and R^{b} are both —H, and Z is selected from the formulae defined in (i) to (ix) above. In certain embodiments R^{a} and R^{b} are both —H, and Z conforms to formula (i) above. In certain embodiments R^{a} and R^{b} are both —H, and Z conforms to formula (ii) above. In certain embodiments R^{a} and R^{b} are both —H, and Z conforms to formula (iii) above. In certain embodiments R^{a} and R^{b} are both —H, and Z conforms to formula (iv) above. In certain embodiments R^{a} and R^{b} are both —H, and Z conforms to formula (v) above. In certain embodiments R^{a} and R^{b} are both —H, and Z conforms to formula (vi) above. In certain embodiments R^{a} and R^{b} are both —H, and Z conforms to formula (vii) above. In certain embodiments R^{a} and R^{b} are both —H, and Z conforms to formula (viii) above. In certain embodiments R^{a} and R^{b} are both —H, and Z conforms to formula (ix) above. In certain embodiments R^{a} and R^{b} are both —H, and Z conforms to formula (x) above. In certain embodiments R^{a} and R^{b} are both —H, and Z conforms to formula (xi) above. In certain embodiments R^{a} and R^{b} are both —H, and Z conforms to formula (xii) above. In certain embodiments R^{a} and R^{b} are both —H, and Z conforms to formula (xiii) above. In certain embodiments R^{a} and R^{b} are both —H, and Z conforms to formula (xiv) above. In certain embodiments R^{a} and R^{b} are both —H, and Z conforms to formula (xv) above. In certain embodiments R^{a} and R^{b} are both —H, and Z conforms to formula (xvi) above. In certain embodiments R^{a} and R^{b} are both —H, and Z conforms to formula (xvii) above.

In certain embodiments, R² is —H. In certain embodiments, R² is a metal cation. In certain embodiments, R² is an organic cation (e.g., a nitrogen or phosphorous centered cationic group). In certain embodiments, R² is an optionally substituted C₁₋₂₀ aliphatic group. In certain embodiments, R² is an optionally substituted C₁₋₁₂ aliphatic group. In certain embodiments, R² is an optionally substituted C₁₋₈ aliphatic group. In certain embodiments, R² is an optionally substituted C₁₋₆ aliphatic group. In certain embodiments, R² is selected from methyl, ethyl, n-propyl, i-propyl, n-butyl, sec-butyl, and t-butyl. In certain embodiments, R² is an optionally substituted aromatic group. In certain embodiments, R² is a glycoside. In certain embodiments, R² comprises an amino acid. In certain embodiments, R² comprises a peptide. In certain embodiments, R² comprises a nucleotide.

In certain embodiments, at least one R³ is —H. In certain embodiments, both R³ groups are —H. In certain embodiments, at least one R³ is an optionally substituted C₁₋₂₀ aliphatic group. In certain embodiments, both R³ groups are an optionally substituted C₁₋₂₀ aliphatic group which may be the same or different. In certain embodiments, at least one R³ is an optionally substituted C₁₋₁₂ aliphatic group. In certain embodiments, at least one R³ is an optionally substituted C₁₋₈ aliphatic group. In certain embodiments, at least one R³ is an optionally substituted C₁₋₆ aliphatic group. In certain embodiments, at least one R³ is selected from methyl, ethyl, n-propyl, i-propyl, n-butyl, sec-butyl, and t-butyl. In certain embodiments, at least one R³ is —CH₂CH₂OH. In certain embodiments, at least one R³ is —CH₂CH₂OR². where R² is as defined in the genera and subgenera herein. In certain embodiments, at least one R³ is an optionally substituted aromatic group. In certain embodiments, at least one R³ comprises a glycoside. In certain embodiments, at least one R³ comprises an amino acid. In certain embodiments, at least one R³ at least one R³ comprises a peptide. In certain embodiments, at least one R³ comprises a nucleotide.

In certain embodiments, an ascaroside is selected from the group consisting of:

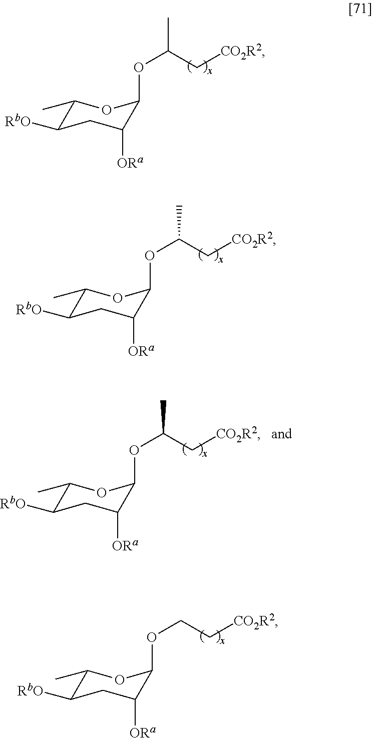

where x is an integer from 1 to 22, and each of R^{a}, R^{b}, and R² is as defined above and in the genera and subgenera herein.

In certain embodiments, an ascaroside is selected from the group consisting of:

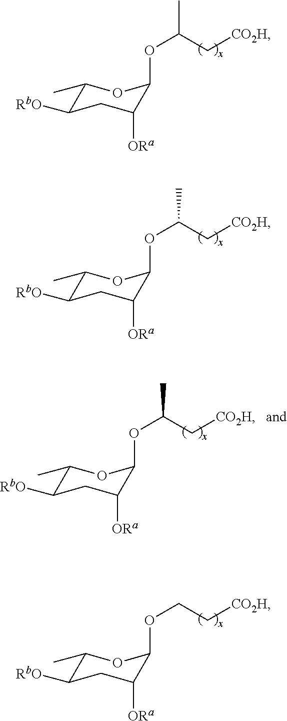

where each of x, R^{a}, and R^{b} is as defined above and in the genera and subgenera herein.

In certain embodiments, an ascaroside is selected from the group consisting of:

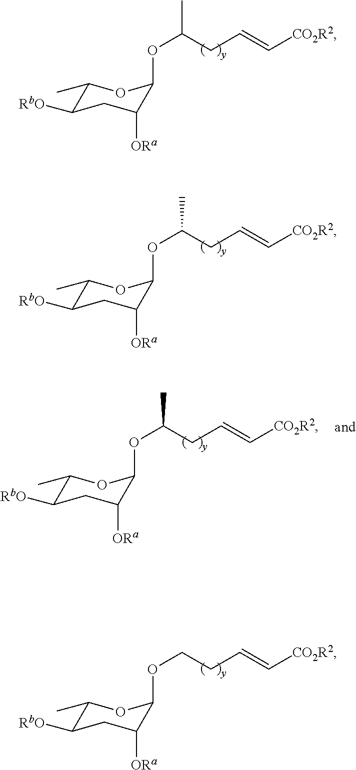

where y is an integer from 1 to 20, and each of R^{a}, R^{b}, and R² is as defined above and in the genera and subgenera herein.

In certain embodiments, an ascaroside is selected from the group consisting of:

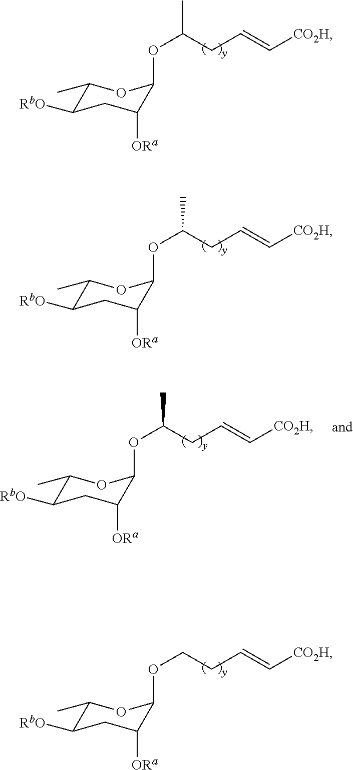

where each of y, R^{a}, and R^{b}, is as defined above and in the genera and subgenera herein.

In certain embodiments, an ascaroside is selected from the group consisting of:

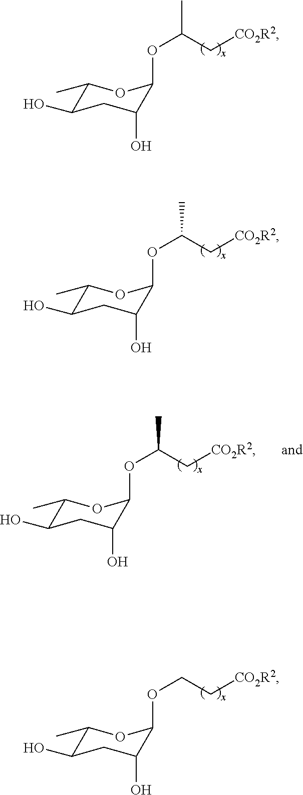

where x is an integer from 1 to 22, and R² is as defined above and in the genera and subgenera herein.

In certain embodiments, an ascaroside is selected from the group consisting of:

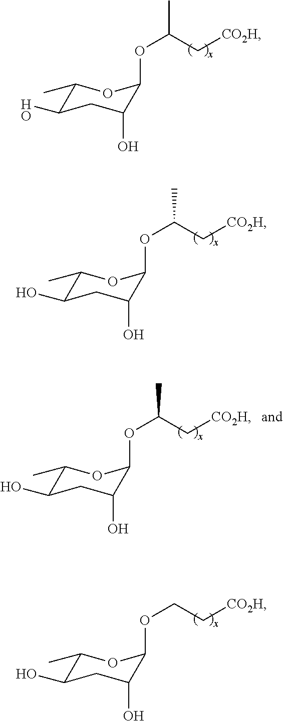

where x is as defined above and in the genera and subgenera herein.

In certain embodiments, an ascaroside is selected from the group consisting of:

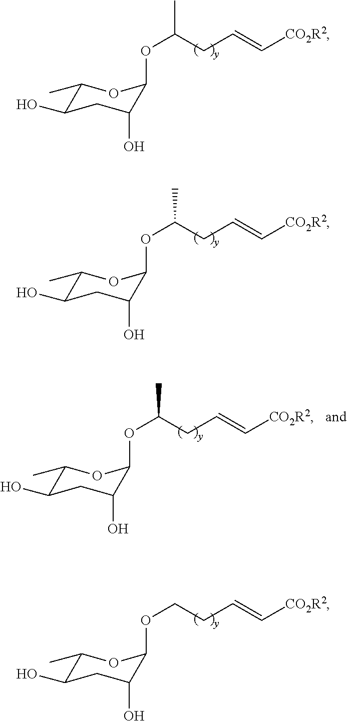

where y is an integer from 1 to 20, and R² is as defined above and in the genera and subgenera herein.

In certain embodiments, an ascaroside is selected from the group consisting of:

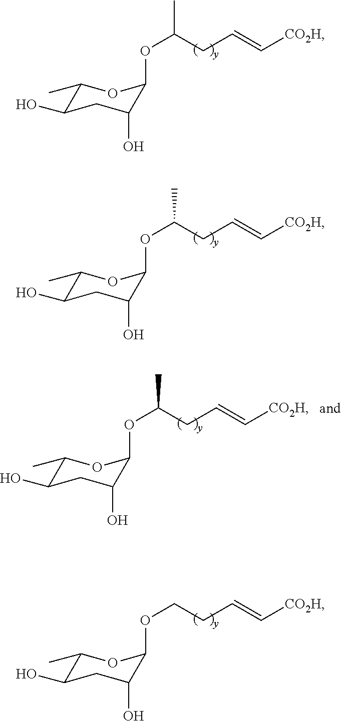

where y is as defined above and in the genera and subgenera herein.

In certain embodiments, an ascaroside is selected from the group consisting of:

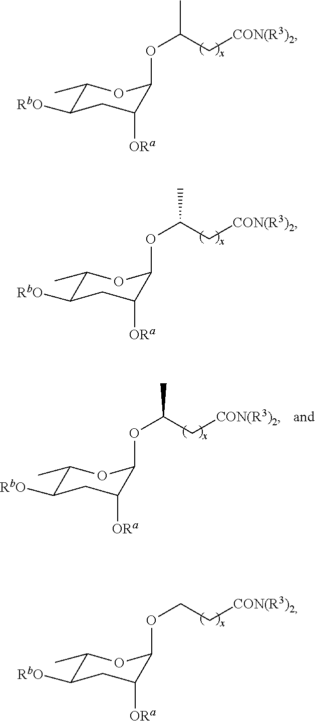

where x is an integer from 1 to 22, and each of R^{a}, R^{b}, and R³ is as defined above and in the genera and subgenera herein.

In certain embodiments, an ascaroside is selected from the group consisting of:

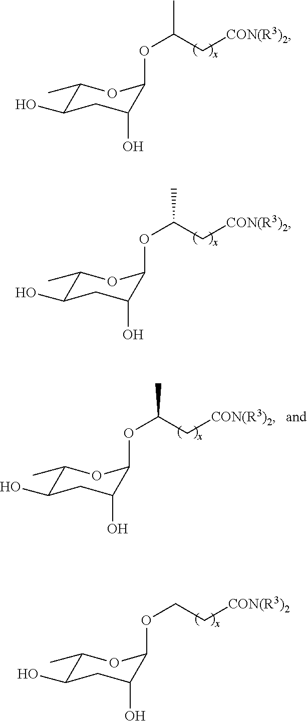

where each of x and R³ is as defined above and in the genera and subgenera herein.

In certain embodiments, an ascaroside is selected from the group consisting of:

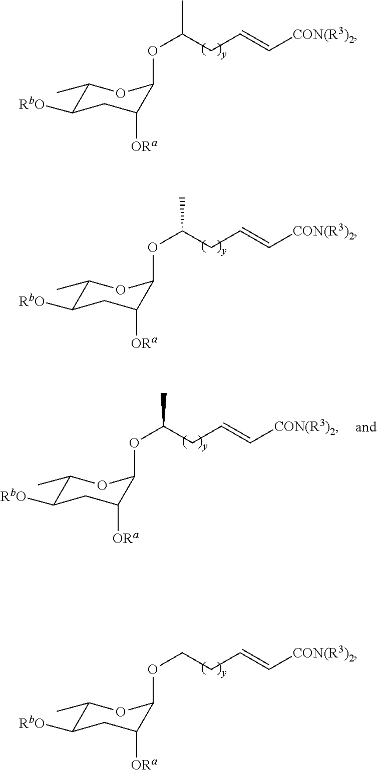

where y is an integer from 1 to 20, and each of R^{a}, R^{b}, and R² is as defined above and in the genera and subgenera herein.

In certain embodiments, an ascaroside is selected from the group consisting of:

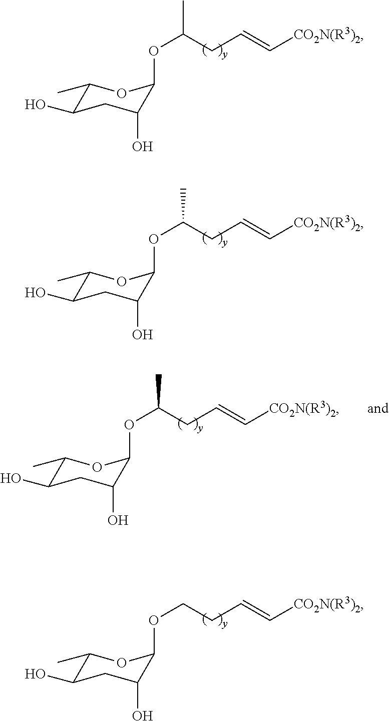

where each of y and R³ is as defined above and in the genera and subgenera herein.

In an embodiment, ascarosides useful in the context of the present disclosure have the general structure (I), where Z is —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide, and can be used for inhibiting human pathogenic bacterial growth in or on a plant.

In an embodiment, ascarosides useful in the context of the present disclosure have the general structure (I) where Z is —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

In an embodiment, ascarosides useful for the present invention have the general structure (I), where Z is —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide, can be used for inhibiting pathogenic bacterial growth in or on a plant.

In an embodiment, ascarosides useful for the present invention have the general structure (I) where Z is —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

Specific ascarosides that are useful for the present invention include, but are not limited to, ascr #7 and ascr #18.

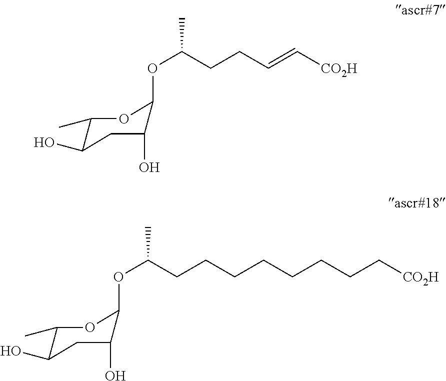 [SMILES: CC1OC(O[C@H](C)CC/C=C/C(=O)O)C(O)CC1O ; CC1OC(O[C@H](C)CCCCCCCCC(=O)O)C(O)CC1O]

In certain embodiments, an ascaroside used in the provided salts, compositions, and/or methods is selected from the group consisting of: ascr #9, ascr #12, ascr #14, ascr #1, ascr #10, ascr #16, ascr #18, ascr #20, ascr #22, ascr #24, ascr #26, ascr #28, ascr #30, ascr #32, ascr #34, and ascr #36. In certain embodiments, an ascaroside used in the provided salts, compositions, and/or methods is selected from the group consisting of: ascr #10, ascr #16, ascr #18, ascr #20, ascr #22, and ascr #24. In certain embodiments, an ascaroside used in the provided methods is selected from the group consisting of: ascr #9, ascr #14, ascr #10, and ascr #18.

In certain embodiments, an ascaroside used in the provided salts, compositions, and/or methods is selected from the group consisting of: ascr #5, oscr #9, oscr #12, oscr #1, oscr #14, oscr #10, oscr #16, oscr #18, oscr #20, oscr #22, oscr #24, oscr #26, oscr #28, oscr #30, oscr #32, oscr #34, and oscr #36. In certain embodiments, an ascaroside used in the provided salts, compositions, and/or methods is selected from the group consisting of: oscr #10, oscr #16, oscr #18, oscr #20, and oscr #22. In certain embodiments, an ascaroside used in the provided salts, compositions, and/or methods is selected from the group consisting of: bhas #5, oscr #9, oscr #12, oscr #1, oscr #14, oscr #10, oscr #16, oscr #18, oscr #20, oscr #22, oscr #24, oscr #26, oscr #28, oscr #30, oscr #32, oscr #34, and oscr #36. In certain embodiments, an ascaroside used in the provided salts, compositions, and/or methods is selected from the group consisting of: oscr #10, oscr #16, oscr #18, oscr #20, and oscr #22.

In certain embodiments, an ascaroside used in the provided salts, compositions, and/or methods is selected from the group consisting of: bhas #9, bhas #10, bhas #16, bhas #18, bhas #22, bhas #24, bhas #26, bhas #28, bhas #30, bhas #32, bhas #34, bhas #36, bhas #38, bhas #40, and bhas #42.

In certain embodiments, an ascaroside used in the provided salts, compositions, and/or methods is selected from the group consisting of: bhos #10, bhos #16, bhos #18, bhos #22, bhos #24, bhos #26, bhos #28, bhos #30, bhos #32, bhos #34, bhos #36, bhos #38, bhos #40, and bhos #42.

In certain embodiments, an ascaroside used in the provided salts, compositions, and/or methods is selected from the group consisting of: ascr #18, oscr #16, oscr #17, oscr #15, bhas #18, bhos #16, glas #18, dhas #18, ibha #18, ibho #16, icas #18, icos #15, icos #16, and any combination of two or more of these.

Ascarosides can be obtained from natural sources (e.g., nematodes) or they may be prepared synthetically. Ascarosides can be prepared synthetically, for example, by converting 1-O-substituted rhamnose to 1-O-substituted ascarylose. An exemplary method of preparing ascarosides includes: providing as a feedstock a 1-O-substituted rhamnose; forming a mono-sulfonate ester at the 3-OH group of the feedstock; and treating the mono-sulfonate ester with a hydride source to form a 1-O-substituted ascarylose. In certain embodiments, forming the mono-sulfonate ester is conducted on a substrate without hydroxyl protecting groups at the 2- or 4-position of the rhamnose feedstock. In certain embodiments, such methods comprise contacting the feedstock with a sulfonating agent (i.e., a sulfonyl halide, sulfonic anhydride or similar reagent) in the presence of a Lewis acid. Specific details regarding the synthesis of 1-O-substituted ascarylose can be found in PCT Application No. PCT/IB2021/056981, which is incorporated herein by reference.

As noted herein above, the present disclosure provides ascarosides in salt form. Ascaroside salts can be formed with appropriate reagents, e.g., at any suitable acidic or basic functional group on the ascaroside molecule.

For example, in some embodiments, carboxylic acid salts (also referred to as carboxylate salts) are provided. Such salts can be depicted according to Formula II, below, where “M” is a metal (or other cation) and “A” is the remainder of the ascaroside molecule to which the carboxylic acid moiety is attached (e.g., the structures according to Formula I, provided herein above):

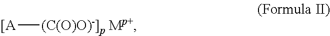

where p represents an integer from 1 to 4. In some embodiments, p is 1 and the salt comprises one ascaroside anion and one “M” cation (with +1 charge). In some embodiments, p is 2 and the salt comprises two ascaroside anions and one “M” cation (with +2 charge). In some embodiments, p is 3 and the salt comprises three ascaroside anions and one “M” cation (with +3 charge). In some embodiments, p is 4 and the salt comprises four ascaroside anions and one “M” cation (with +4 charge). In embodiments wherein p is greater than 1, the ascaroside anions present in the salt can be the same or different.

Referring to the structure of the ascaroside in Formula I, above, it is noted that a carboxylic acid salt can be formed, e.g., at the “OR^{a}” and/or “OR^{b}” substituent of the tetrahydropyranyl ring. In some embodiments, a carboxylic acid can be formed at the ORₐ and/or OR_{b} substituent where Rₐ and/or R_{b} is -L-C(O)OR^{c}, where L is an optionally-substituted bivalent linker comprising one or more carbon atoms and optionally containing one or more heteroatoms and where R^{c} is H. In some embodiments, a carboxylic acid can be formed at the OR^{a} and/or OR^{b} substituent where R^{a} and/or R^{b} is a sugar moiety (e.g., a sugar acid) comprising a carboxylic acid group. In some embodiments, a carboxylic acid can be formed at the OR^{a} and/or OR^{b} substituent where R^{a} and/or R^{b} is a peptide comprising a carboxylic acid group. In some embodiments, a carboxylic acid can be formed at the OR^{a} and/or OR^{b} substituent where R^{a} and/or R^{b} is a polymer comprising a carboxylic acid group. In some embodiments, a carboxylic acid can be formed at the OR^{a} and/or OR^{b} substituent where R^{c} is a polymer chain comprising a carboxylic acid group. In some embodiments, a carboxylic acid can be formed at the OR^{a} and/or OR^{b} substituent where Rₐ and/or R^{b} is a mono-ester of a diacid. In certain embodiments, such compounds are derived from reaction of a compound where R^{a} and/or R^{b} is —H with a cyclic acid anhydride such as phthalic, succinic, maleic or glutaric anhydride or any of their substituted analogs. Representative examples of such ascaroside anions are shown below:

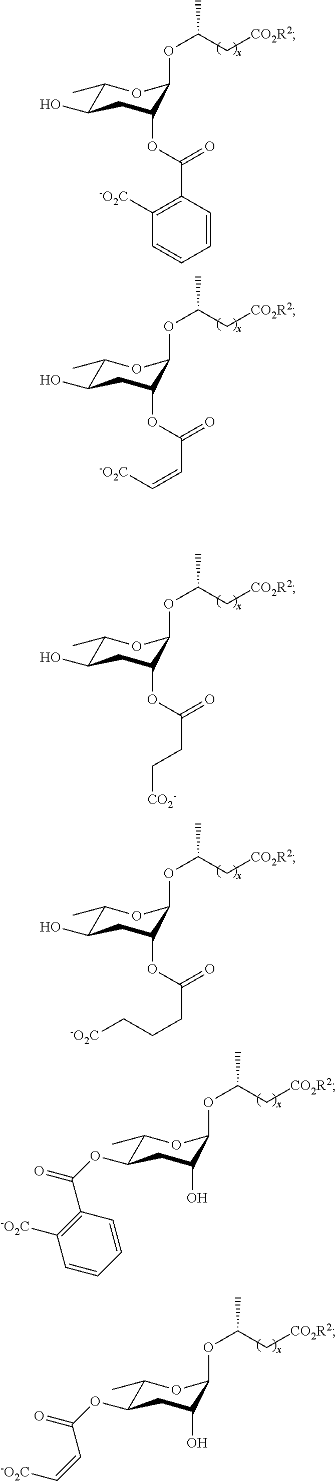

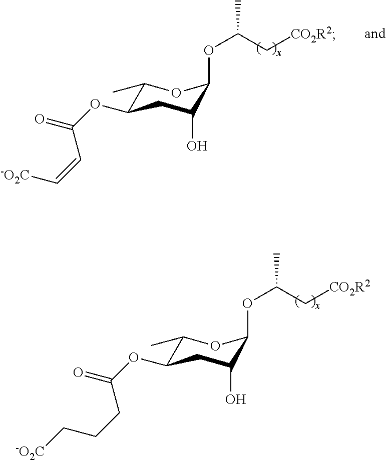

where each of x and R² is as defined above and in the genera and subgenera herein.

Again referring to the structure of the ascaroside in Formula I, above, it is noted that a carboxylic acid can be formed, e.g., at the OZ substituent of the tetrahydropyranyl ring. In some embodiments, a carboxylic acid can be formed at the OZ substituent, e.g., when Z is selected from —CH(CH₃)—(CH₂)ₙ—CO₂R²; —CH(CH₃)—(CH₂)ₙ—CH═CH—CO₂R², —CH(CH₃)—(CH₂)ₙ—CH(OH)—CH—CO₂R²; —CH(CH₃)—(CH₂)ₙ—C(O)—CH—CO₂R²; —(CH₂)ₙ—CO₂R²; —(CH₂)ₙ—CH═CH—CO₂R²; and —(CH₂)ₙ—CH(OH)—CH—CO₂R², where n is an integer from 1 to 40 and R² is —H or an optionally substituted C₁₋₂₀ aliphatic group, optionally substituted aromatic group, a glycoside, amino acid, a peptide, or nucleotide that contains a carboxylic acid group.

In certain embodiments, a linker group -L- comprises an optionally substituted C₁₋₂₀ aliphatic, C₁₋₂₀ heteroaliphatic, aryl, or heteroaryl. Such linkers may be attached to an oxygen atom on the ascaroside molecule via an ether linkage, an ester linkage, a carbonate linkage, a carbamate linkage (e.g., A-OC(O)NR³—), a thioester linkage (e.g., A-OC(S)—), a thiocarbamate linkage (e.g., A-OC(S)NR³—), a thiocarbonate linkage (e.g., A-OC(S)O—), a dithiocarbonate linkage (a thiocarbonate linkage (e.g., A-OC(S)S—), a sulfonate ester linkage (e.g., A-OSO₂—), or via a linkage through a phosphorus atom.

In one embodiment, a salt of ascr #18 is provided, e.g., according to the following formula:

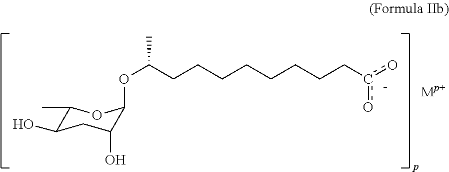

where negative charge is shown delocalized over the oxygen atoms of the carboxylic acid.

The disclosure is not limited to carboxylic acid salts of ascarosides. Salts that can be formed with other functional groups on an ascaroside molecule are also encompassed within the present disclosure. For example, where an ascaroside comprises an amine group (e.g., within the R^{a}, R^{b}, or Z substituent), an ammonium salt can be formed with suitable anionic groups. Further, in some embodiments, where an ascaroside comprises a hydroxyl (—OH) group, certain M groups may coordinate thereto to form a salt, e.g., as in a borate or silicate derivative of an ascaroside.

Shown below are non-limiting, representative metal salts of ascarosides formed through the carboxylic acid group of the sidechain:

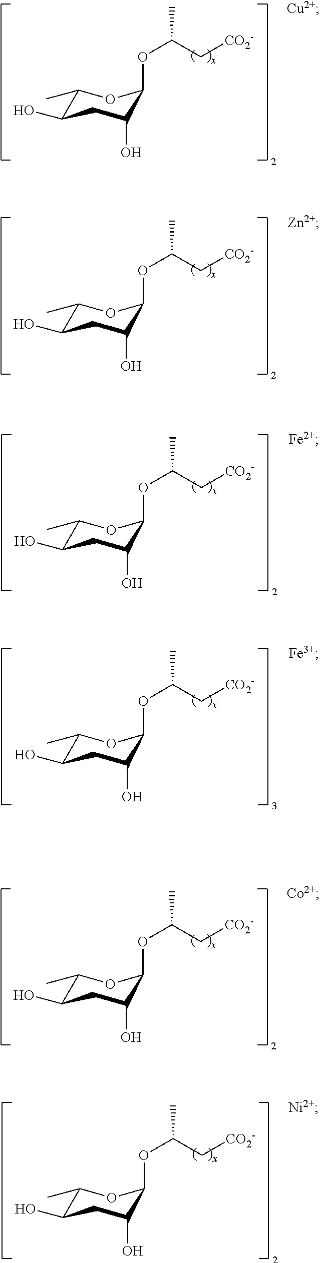

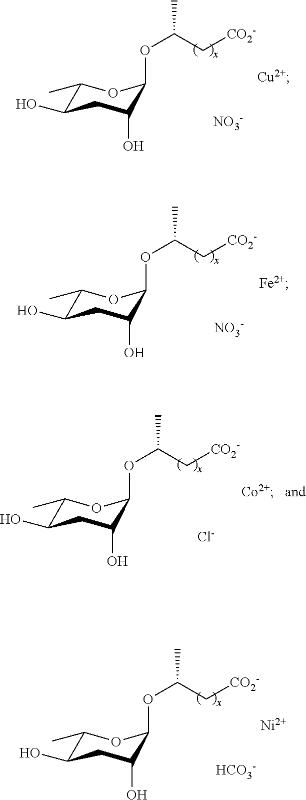

where x is as defined above and in the genera and subgenera herein.

Shown below are representative “ate” salts of ascarosides formed through the hydroxy groups of the ascarylose sugar:

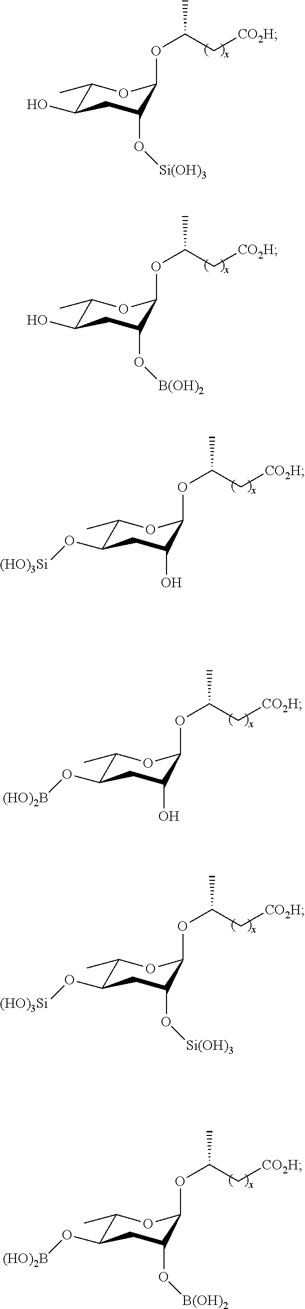

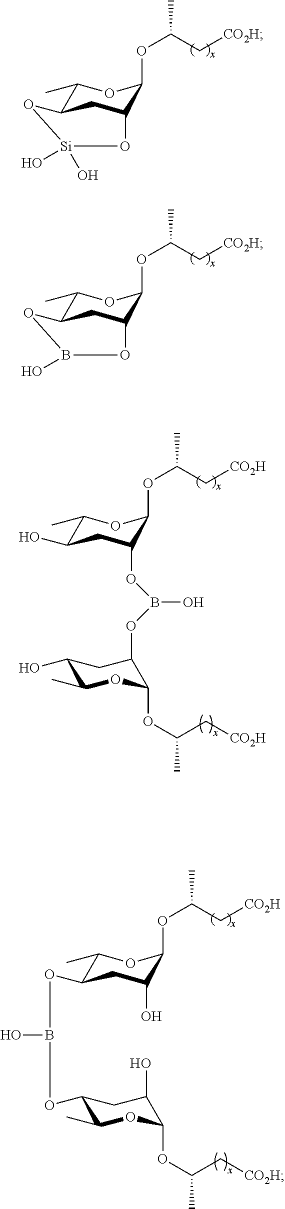

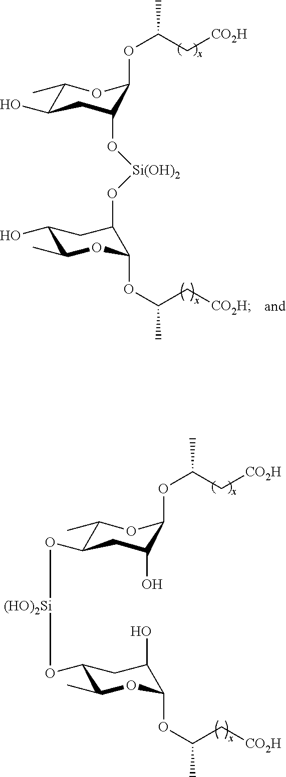

where x is as defined above and in the genera and subgenera herein.

The composition of “M” (e.g., as shown in Formula II and IIb above) can vary widely. In some embodiments, M is a metal. Suitable metals are not particularly limited and, in various embodiments, are selected from alkali/Group 1 metals, alkaline earth/Group 2 metals, and transition metals. In some embodiments, M is a semi-metal or a non-metal, selected from the main group elements. In some embodiments, M is an organic cation such as an ammonium, guanidinium, amidinium, pyridinium, phosphonium, sulfonium, or any similar nitrogen-, phosphorus- or sulfur-centered cation or analog thereof.

In some embodiments, M comprises a plant micronutrient or a plant macronutrient. Plant micronutrients and macronutrients include, but are not limited to, nitrogen (N), phosphorus (P), potassium (K), calcium (Ca), magnesium (Mg), boron (B), silicon (Si), zinc (Zn), manganese (Mn), iron (Fe), copper (Cu), molybdenum (Mo), and nickel (Ni). One of skill in the art will appreciate that not all such micronutrients and macronutrients can form salts with ascarosides in the same manner.

For example, salts of plant nutrients such as potassium, calcium, magnesium, zinc, manganese, iron, copper, molybdenum, and nickel can, in some embodiments, be formed with a carboxylic acid on an ascaroside (e.g., according to Formula II or IIb above). Nitrogen may be provided in the form of an ammonium or guanidinium salt of an ascaroside (e.g., wherein M is NH₄^{|} or H₂N^{|}=C(NH₂)₂ or an ammonium or guanidinium analogue salt of an ascaroside (e.g., wherein one or more of the H atoms of NH₄⁺ or H₂N⁺=C(NH₂)₂ is replaced with an alkyl group). Plant nutrients such as boron, silicon and molybdenum may not easily form salts with carboxylic acids but may, for example, coordinate in the form of a borate, a silicate, or a molybdate to a free OH present on the ascaroside molecule to form a suitable salt. As such, in some embodiments, a salt comprising at least one boron atom coordinated to an ascaroside, a salt comprising at least one silicon atom coordinated to an ascaroside, or a salt comprising at least one molybdenum atom coordinated to an ascaroside is provided.

The salts provided herein can be prepared by adapting conventional methods known in the art. For example, a salt is frequently the result of reaction of an acid and a base. As such, an ascaroside salt as provided above in Formula II or IIb can be prepared, for example, by combining one or more ascaroside molecules (the number of molecules corresponding to the number “p,” i.e., equivalent to the charge on the “M” (e.g., metal or ammonium cation)) with MP+in solution at a pH sufficient to afford the desired salt. In other embodiments, salts may be produced by metathesis, where the cation and/or anion in a first salt is replaced by a different cation or anion from a second salt.

The ascaroside salts disclosed herein can, in some embodiments, exhibit enhanced physical properties as compared with a corresponding ascaroside molecule. For example, in some embodiments, ascaroside salts can exhibit greater solubility in certain solvents (e.g., water) than the corresponding ascarosides, thus simplifying the preparation of certain formulations. In some embodiments, providing an ascaroside in salt form (rather than in its neutral/free acid or base form) renders it more compatible with certain components of a desired composition, thus stabilizing the resulting ascaroside-containing composition.

In some embodiments, e.g., where M is a plant micronutrient or macronutrient, an ascaroside salt as provided herein provides advantages beyond formulation benefits, e.g., providing nutrients to a plant to which (or near which) it is applied. As such, in some embodiments, salts provided herein can be referred to as ascaroside-based micronutrients or ascaroside-based macronutrients. In some embodiments, the salts described herein have improved application characteristics (e.g., enhanced ability to wet or adhere to surfaces such as leaves or skin), enhanced bioavailability, and/or enhanced uptake (e.g., by a plant, microorganism, or animal such as a mammal).

In certain embodiments, ascaroside salts and ascaroside salt-containing compositions provided herein comprise “multifunctional” products. In addition to providing nutrients as described above, in certain embodiments, such multifunctional products combine ascarosides with molecules having other different, or complementary biological activity. In certain embodiments, such multifunctional products comprise a salt formed from the anion of an ascaroside with a cation of a molecule having bioactivity as a pesticide (e.g., insecticides, fungicides, herbicides, miticides, nematicides), as an antimicrobial agent (e.g., antibiotics, antifungals, etc.), as antiviral agents, as signaling molecules (e.g., pheromones, hormones, etc.), or as vitamins or nutrients. Such multifunctional products can avoid the need for multiple applications (for example of a drug or an agricultural treatment), thereby saving costs and/or simplifying methods of treatment.

In certain embodiments, salts or compositions provided herein comprise a salt of an ascaroside anion with a cationic pesticide molecule. In certain embodiments, salts or compositions provided herein comprise a salt of an ascaroside anion with a cationic insecticide molecule. In certain embodiments, salts or compositions provided herein comprise a salt of an ascaroside anion with a cationic herbicide molecule. In certain embodiments, salts or compositions provided herein comprise a salt of an ascaroside anion with a cationic fungicide molecule. In certain embodiments, salts or compositions provided herein comprise a salt of an ascaroside anion with a cationic antimicrobial molecule. The identification (and provision or synthesis) of suitable bioactive cationic molecules is within the grasp of the skilled artisan as is the formation of such salts using straightforward acid-base chemistry or ion metathesis techniques.

In certain embodiments, salts or compositions provided herein comprise a salt of an ascaroside anion with a cationic signaling molecule. In certain embodiments, salts or compositions provided herein comprise a salt of an ascaroside anion with a cationic plant hormone. In certain embodiments, salts or compositions provided herein comprise a salt of an ascaroside anion with a cationic pheromone.

In certain embodiments, salts or compositions provided herein comprise a salt of an ascaroside anion with a cationic therapeutic molecule. In certain embodiments, salts or compositions provided herein comprise a salt of an ascaroside anion with a cationic vitamin. In certain embodiments, salts or compositions provided herein comprise a salt of an ascaroside anion with a cationic pharmaceutical. In certain embodiments, salts or compositions provided herein comprise a salt of an ascaroside anion with a cationic hormone. In certain embodiments, salts or compositions provided herein comprise a salt of an ascaroside anion with a cationic vitamin.

In certain embodiments, the ascaroside salts described herein can find use in treating living plants, soil surrounding plants, or soil in which seeds/seedlings are to be planted. In some embodiments, the ascaroside salt (or a composition comprising such ascaroside salt) is applied to a portion of a plant, e.g., one or more of a root, stem, leaf, seed, and/or flower. Such methods can be conducted at any one or more stages in the life cycle of a plant, e.g., from seed to seedling to growing plant to just prior to harvest.

In certain embodiments where the provided ascaroside salt incorporates a second bioactive molecule, the second bioactive molecule may be an anion, and the composition may comprise a mixed salt where the ascaroside and the second bioactive anionic molecule share a common cation. In certain embodiments, such mixed salts can comprise a polycation capable of simultaneously forming a salt with the ascaroside and the second bioactive anionic molecule. In certain embodiments, such mixed salts can comprise a common mono-cationic species or mixture of mono cations that balance the anionic charge of both the ascaroside and the second bioactive anionic molecule.

The disclosed treatment methods can, in some embodiments, protect growing plants in the manner described in U.S. Pat. No. 10,136,595, which is incorporated by reference herein in its entirety. For example, such methods can enhance pathogen resistance and/or induce one or more plant defense responses (thereby inhibiting pathogen growth and/or infestation) in a plant to (or near) which the ascaroside salt (or composition comprising such ascaroside salt) is applied. Pathogens against which the disclosed methods can enhance resistance include, but are not limited to, oomycetes, bacteria, nematodes, viruses, and insects, e.g., including but not limited to, Pseudomonas syringae, Phytophthora infestans, Blumeria graminis, Heterodera schachtii, Meloidogyne incognita, Meloidogyne hapla, and turnip crinkle virus.

The disclosed treatment methods can, in some embodiments, protect plants or plant-based products from contamination with pathogens or prevent or delay the onset of spoilage of plants or produce in the manner described in patent applications PCT/US22/81895 and PCT/US23/17947 each of which is incorporated by reference herein in its entirety.

The exact method by which a plant or soil is treated with an ascaroside salt or Ascaroside salt-containing composition is not particularly limited. Treatment of plants and/or soil according to the present disclosure can be carried out, e.g., by immersion, spraying, cvaporation, fogging, scattering, painting on, side dressing, or in-furrow application. For example, in certain embodiments, plants or soil can be sprayed with a suitable liquid composition, a solid plastic mulch composition can be applied on soil around plants, and/or a granular composition can be provided for in-furrow application or side-dressing.

The types of plants that can be treated according to the presently disclosed methods is not particularly limited and can be, for example, fruit and vegetable plants, trees, and shrubs. Non-limiting examples of plants that can be treated according to the disclosed methods include, but are not limited to, plants selected from the group consisting of tobacco, Arabidopsis, tomato, barley, potato, sweet potato, yam, cotton, soybean, strawberry, sugar beet, corn, rice, wheat, rye, oat, sorghum, millet, bean, pea, apple, banana, pear, cherry, peach, plum, apricot, almond, grape, kiwi, mango, melon, papaya, walnut, hazelnut, pistachio, raspberry, blackberry, loganberry, blueberry, cranberry, orange, lemon, grapefruit, tangerine, lettuce, carrots, onions, broccoli, cabbage, avocado, cocoa, cassava, cotton, and flax.

In the methods provided herein, ascaroside salts can be directly applied to the plant and/or soil or can be formulated into a composition that can be applied to the plant and/or soil. As such, the present disclosure provides compositions that generally comprise at least one ascaroside salt and one or more inert ingredients, e.g., one or more agronomically acceptable carriers. It is preferred that non-toxic carriers be used in the methods of the present disclosure.

The term “agronomically acceptable carrier” includes any carrier suitable for administration to a plant or soil, e.g., customary excipients in formulation techniques, such as used to form solutions (e.g., directly sprayable or dilutable solutions), emulsions, (e.g., emulsion concentrates and diluted emulsions), wettable powders, suspensions, soluble powders, powders, dusts, pastes, soluble powders, granules, suspension-emulsion concentrates, encapsulation into polymeric materials, coatable pastes, natural and synthetic materials impregnated with active compound/salt and microencapsulations in polymeric substances. These compositions can be produced in a known manner, for example, by mixing the ascaroside salt(s) with one or more agronomically acceptable carriers, such as liquid solvents or solid carriers, optionally with the use of additional components including, but not limited to, surfactants, including emulsifiers, dispersants, foam-formers, colorants, processing aids, lubricants, fillers, reinforcements, flame retardants, light stabilizers, ultraviolet radiation absorbers, weather stabilizers, plasticizers, release agents, perfumes, heat-retaining additives (e.g., silica), cross-linking agents, antioxidants, anti-foaming agents, buffers, pH modifiers, compatibility agents, drift control additives, extenders/stickers, tackifiers, plant penetrants, safeners, spreaders, wetting agents, and the like.

In some embodiments, the ascaroside salt is the only active agent within the composition. In some embodiments, the composition includes one or more additional ascarosides (e.g., in another salt form or in a non-salt form). In some embodiments, one or more other active agents are included within the composition (e.g., one or more pesticides (e.g., insecticides, fungicides, herbicides, miticides, nematicides), antimicrobial agents (e.g., antibiotics, antifungals, etc.), antiviral agents, signaling molecules (e.g., pheromones, hormones, etc.), vitamins, nutrients, or fertilizers, etc.). The ascaroside salt-containing compositions provided herein can be in various forms, including solid and liquid forms.

If the agronomically acceptable carrier is water, in some embodiments, an organic solvent may optionally be incorporated as an auxiliary liquid solvent. Suitable liquid solvents include, for example, aromatics (e.g., xylene, toluene and alkylnaphthalenes); chlorinated aromatics or chlorinated aliphatic hydrocarbons (e.g., chlorobenzenes, chloroethylenes and methylene chloride); aliphatic hydrocarbons (e.g., cyclohexane); paraffins (e.g., petroleum fractions, mineral and vegetable oils); alcohols (e.g., ethanol, butanol or glycol and their ethers and esters, including propylene glycol); ketones (e.g., acetone, methyl ethyl ketone, methyl isobutyl ketone and cyclohexanone) and strongly polar solvents (e.g., dimethylformamide and dimethyl sulfoxide).

Suitable solid agronomically acceptable carriers include, for example, ammonium salts and ground natural minerals (e.g., kaolins, clays, talc, chalk, quartz, attapulgite, montmorillonite and diatomaceous earth); ground synthetic minerals (e.g., highly disperse silica, alumina and silicates); crushed and fractionated natural rocks (e.g., calcite, marble, pumice, sepiolite and dolomite); synthetic granules of inorganic and organic meals; and/or granules of organic material (e.g., sawdust, coconut shells, maize cobs and tobacco stalks).

Suitable emulsifiers and foam-formers include, for example, nonionic and anionic emulsifiers (e.g., polyoxyethylene fatty acid esters, polyoxyethylene fatty alcohol ethers, for example, alkylaryl polyglycol ethers, alkylsulfonates, alkyl sulphates and arylsulfonates) and protein hydrolysates.

Suitable dispersants include, for example, lignin-sulfite waste liquors and methylcellulose. Tackifiers such as carboxymethylcellulose and natural and synthetic polymers in the form of powders, granules or lattices, such as gum arabic, polyvinyl alcohol and polyvinyl acetate, as well as natural phospholipids, such as cephalins and lecithins, and synthetic phospholipids, can be used in the disclosed compositions. Other additives may include, for example, mineral and vegetable oils.

Colorants such as inorganic pigments, for example, iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc may also be included in the compositions.

Methods of preparing solid and liquid compositions for agrichemical use are generally known and can be employed according to the present disclosure (where such methods involve incorporating one or more ascaroside salts within such compositions). Compositions according to the present disclosure can, in some embodiments, be in the form of granular material (including dusts, pellets, soluble powders, flowable powders, water-dispersible granules, and the like). In some embodiments, compositions according to the present disclosure can be in liquid form (e.g., solutions, suspensions, concentrates or emulsions). In some embodiments, compositions are in the form of a granular material treated with an ascaroside salt-containing liquid. In some embodiments, a composition comprising an ascaroside salt is formed into fibers or filaments and in some such embodiments, a woven or non-woven textile (e.g., film) can be produced therefrom. In some embodiments, a composition as provided herein is pelletized. In some embodiments, a composition as provided herein is in the form of a film, e.g., plastic mulch. Any of the solid compositions provided herein can optionally be coated via methods generally known in the art to delay release of the ascaroside salt. See, e.g., the methods and compositions for modified release disclosed in PCT/US2023/20472, filed Apr. 28, 2023, which is incorporated herein by reference.

It is contemplated that compounds (including salts), compositions, and methods of the present application encompass variations and adaptations developed using information from the embodiments described in the present disclosure. Adaptation or modification of the methods and processes described in this specification may be performed by those of ordinary skill in the relevant art.

It will be appreciated that use of headers in the present disclosure are provided for the convenience of the reader. The presence and/or placement of a header is not intended to limit the scope of the subject matter described herein. Unless otherwise specified, embodiments located in one section of the application apply throughout the application to other embodiments, both singly and in combination.

Throughout the description, where compositions, compounds, or products are described as having, including, or comprising specific components, or where processes and methods are described as having, including, or comprising specific steps, it is contemplated that, additionally, there are articles, devices, and systems of the present application that consist essentially of, or consist of, the recited components, and that there are processes and methods according to the present application that consist essentially of, or consist of, the recited processing steps.

### EXAMPLES

In order that the application may be more fully understood, the following examples are set forth. It should be understood that these examples are for illustrative purposes only and are not to be construed as limiting in any manner.

Various salts of the ascaroside ascr #18 were produced and tested for their ability to protect plants from pathogen damage.

### Preparation of the Salts

To 2.5 mL of a 1 mM solution of ascr #18 in water was added an equal volume of 0.5 mM aqueous zinc acetate. The mixture was stirred for 1 h then evaporated under vacuum to provide (ascr #18)₂Zn as a white solid.

To 2.5 mL of a 1 mM solution of ascr #18 in water was added an equal volume of aqueous 1 mM potassium hydroxide. The mixture was stirred for 1 h then evaporated under vacuum to provide (ascr #18)K as a white solid.

To 2.5 mL of a degassed 1 mM solution of ascr #18 was added an equal volume of degassed 0.5 mM Iron(II) acetate. The mixture was stirred for 1 h under nitrogen, then evaporated under vacuum to provide (ascr #18)₂Fe as a yellow-orange solid which was stored under nitrogen until use.

To 2.5 mL of a 1 mM solution of ascr #18 was added an equal volume of 0.5 mM nickel(II) acetate trihydrate. The mixture was stirred for 1 h then evaporated under vacuum to provide (ascr #18)₂Ni as a greenish solid.

To 2.5 mL of a 1 mM solution of ascr #18 was added an equal volume of 0.5 mM manganese(II) acetate tetrahydrate. The mixture was stirred for 1 h then evaporated under vacuum to provide (ascr #18)₂Mn as a brown solid.

### Testing of the Salts

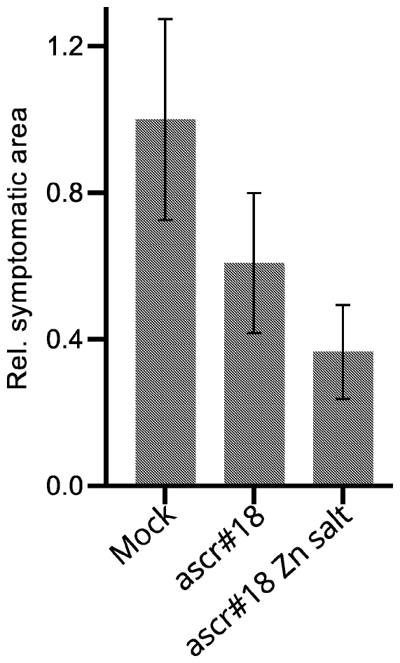

The efficacy of the zinc salt of ascr #18 was compared to free ascr #18 in a greenhouse-based plant pathogen assay. Two-week-old wheat plants were sprayed with a solution containing 1 μM ascr #18, or 1 μM of the zinc salt (ascr #18)₂Zn. 24 h after treatment, the plants were inoculated with spores of Bipolaris sorokiniana (causal agent of root rot and brown spot blotch). Disease symptoms were quantified ten days after inoculation by measuring the symptomatic area on the leaves. As shown in FIG. 1, the zinc salt provided better protection against pathogen damage than un-modified ascr #18 (Data are mean±SEM (n≥13). *P≤0.04; two-tailed t-test).

It should be understood that the order of steps or order for performing certain action is immaterial so long as the described method remains operable. Moreover, two or more steps or actions may be conducted simultaneously.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A salt of the formula [A-(C(O)O)⁻]ₚM^{p+}, wherein:
A is an ascaroside moiety;
M is a metal or ammonium cation; and
p is an integer from 1 to 3,
and wherein, when p is 2 or 3, each A can be the same or different.

2. The salt of claim 1, wherein p is 1.

3. The salt of claim 1, wherein p is 2.

4. The salt of claim 1, wherein p is 3.

5. The salt of any one of claims 1-4, wherein M is a metal.

6. The salt of claim 5, wherein the metal is selected from the group consisting of potassium (K), calcium (Ca), magnesium (Mg), zinc (Zn), manganese (Mn), iron (Fe), copper (Cu), molybdenum (Mo), and nickel (Ni).

7. The salt of claim 6, wherein the metal is selected from the group consisting of copper (Cu), zinc (Zn), manganese (Mn), and iron (Fe).

8. The salt of any one of claims 1-4, wherein M is an ammonium cation or an analogue thereof.

9. A salt comprising at least one boron atom coordinated to an ascaroside (A).

10. The salt of any of claims 1-9, wherein A has the structure (I) 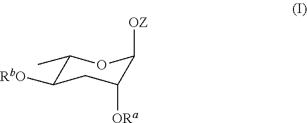
where:
Z is an optionally substituted C₂₋₄₀ aliphatic group, and
each of R^{a} and R^{b} is independently —H, or an optionally substituted moiety selected from the group consisting of: C₁₋₂₀ aliphatic, C₁₋₂₀ acyl, C₁₋₂₀ heteroaliphatic, aryl, heteroaryl, a hydroxyl protecting group, a phosphorous-linked functional group, a sulfur-linked functional group, a silicon-linked functional group, a C₂₋₂₀ carbonate (e.g., a moiety -C(O)OR^{c}), a C₂₋₂₀ carbamate (e.g., a moiety) —C(O)N(R²), a C₂₋₂₀ thioester (e.g., a moiety —C(S)R^{c}), a C₂₋₂₀ thiocarbonate (e.g. a moiety —C(S)OR^{c}), a C₂₋₂₀ dithiocarbonate (e.g., a moiety —C(S)SR^{c}), a C₁₋₂₀ thiocarbamate (e.g., a moiety —C(S)N(R^{c})₂), a sugar moiety, a peptide, a polymer chain, or a linkage via a bond or a carbon-containing linker moiety to another ascaroside molecule, where R^{c} is independently at each occurrence selected from —H, optionally substituted C₁₋₁₂ aliphatic, optionally substituted C₁₋₁₂ heteroaliphatic, optionally substituted aryl, optionally substituted heteroaryl, a polymer chain, or a linkage via a bond or a carbon-containing linker moiety to another ascaroside molecule, and where R^{a} and R^{b} may be taken together to form an optionally substituted ring, optionally containing one or more heteroatoms, and optionally containing one or more sites of unsaturation.

11. The salt of claim 10, wherein Z is selected from the group consisting of:
i. —CH(CH₃)—R¹, where R¹ is an optionally substituted C₁₋₄₀ aliphatic group;
ii. —CH(CH₃)—(CH₂)ₙ—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
iii. —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
iv. —CH(CH₃)—(CH₂)ₙ—CH(OH)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
V. —CH(CH₃)—(CH₂)ₙ—C(O)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
vi. —(CH₂)ₙ—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
vii. —(CH₂)ₙ—CH═CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
viii. —(CH₂)ₙ—CH(OH)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule; and
ix. —(CH₂)ₙ—C(O)—CH—CO₂R₂, where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule.

12. The salt of claim 10 or 11, wherein R^{a} and R^{b} are each —H.

13. The salt of any one of claims 10-12, wherein Z is —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

14. The salt of any one of claims 1 to 13, wherein A is ascr #18.

15. A salt of the formula: 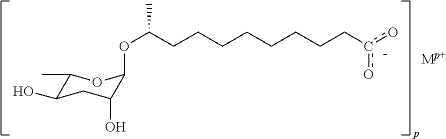
wherein M is a metal or ammonium cation; and
p is an integer from 1 to 3.

16. A composition comprising the salt of any of claims 1-15.

17. The composition of claim 16, in solid form.

18. The composition of claim 17, wherein the solid form comprises powder or granules.

19. The composition of claim 16, in liquid form.

20. The composition of claim 19, wherein the liquid form is a sprayable formulation.

21. The composition of any of claims 16-20, further comprising one or more additional components selected from the group consisting of surfactants, including emulsifiers, dispersants, foam-formers, colorants, processing aids, lubricants, fillers, reinforcements, flame retardants, light stabilizers, ultraviolet radiation absorbers, weather stabilizers, plasticizers, release agents, perfumes, heat-retaining additives (e.g., silica), cross-linking agents, antioxidants, anti-foaming agents, buffers, pH modifiers, compatibility agents, drift control additives, extenders/stickers, tackifiers, plant penetrants, safeners, spreaders, and wetting agents.

22. A method of enhancing the physical properties of an ascaroside and providing one or more nutrients to a plant, comprising: contacting a plant or portion thereof or soil surrounding the plant or portion thereof with the salt of any of claims 1-15 or the composition of any of claims 16-21.

23. The method of claim 22, wherein the portion thereof is selected from the group consisting of root, stem, leaf, seed, and flower.

24. The method of claim 22 or 23, wherein the plant is a vegetable or fruit plant.
